(19) United States
(12) Patent Application Publication
(10) Pub. No.: US 2010/0059376 A1
(43) Pub. Date: Mar. 11, 2010

(54) NOVEL ANALYTICAL METHOD FOR PROTEIN

(75) Inventors: Dong Il Jin, Daejeon (KR); Jae Young Lee, Daejeon (KR); Hong Rae Kim, Seoul (KR); Chang-Sik Park, Chungcheongnam-do (KR)

Correspondence Address: EDWARDS ANGELL PALMER & DODGE LLP, P.O. BOX 55874, BOSTON, MA, 02205, US

(73) Assignee: The Industry & Academic Corporation in Chungnam National University, Daejeon (KR)

(21) Appl. No.: 12/620,463

(22) Filed: Nov. 17, 2009

Related U.S. Application Data
(63) Continuation of application No. PCT/KR2008/003116, filed on Jun. 4, 2008.

(30) Foreign Application Priority Data
Jun. 5, 2007 (KR) 10-2007-0054930

(51) Int. Cl. G01N 33/68; G01N 27/26

(52) U.S. Cl. 204/461

(57) ABSTRACT

Disclosed is a method of analyzing a protein, comprising the steps of: (A) labeling small amounts of protein samples with CyDyes having different fluorescent properties, respectively; (B) mixing 50-100 μg of each of the protein samples, labeled in step (A), with 1-5 mg of each of one or more of the unlabeled protein samples, and subjecting the mixture to electrophoresis; (C) subjecting the electrophoresed gel to fluorescence analysis to detect a difference in protein distribution between the protein samples; and (D) excising spot(s), showing the difference in protein distribution, from the gel, and isolating and identifying from the spots a protein, which is different in protein distribution between the protein samples. According to the present invention, a protein from a gel can be identified by MALDI-TOF, while simultaneously analyzing the gel qualitatively and quantitatively using a difference in the fluorescence of CyDyes with very high sensitivity.

### CROSS-REFERENCE TO RELATED APPLICATION

This application is a continuation of PCT/KR2008/003116 filed on Jun. 4, 2008, which claims priority of Korean Application No. 10-2007-0054930 filed on Jun. 5, 2007, the entire contents of which are incorporated herein by reference.

### TECHNICAL FIELD

The present invention relates to a method for analyzing a protein, and more particularly, a method for analyzing or identifying a protein in a sample using fluorescent dye (e.g., CyDye). The method can simultaneously analyze and more easily compare multiple (e.g., two, three, or more) samples of proteins.

### BACKGROUND ART

2-Dimensional gel electrophoresis, first described in 1975 by O'Farrell, is a common technique for analyzing a protein. The technique separates proteins according to isoelectric point (pI) in a first dimension, and according to molecular weight in a second dimension. Using 2-dimensional gel electrophoresis, proteins contained in a biological sample, (e.g., from a tissue or a cell) can be isolated, and characteristics of the proteins, and their isoforms can be analyzed, such as molecular weight and isoelectric point.

MALDI-TOF (Matrix-Assisted Laser Desorption Ionization-Time of Fight) is a method used in research for polymeric biocompounds that is capable of measuring molecular mass very precisely. Particularly, a protein is subjected to 2-dimensional gel electrophoresis prior to MALDI-TOF analysis. After separating the proteins by 2-dimensional gel electrophoresis, the gel is stained with Coomassie blue, and the resulting protein spots are excised from the gel to isolate a protein, and then the protein is identified by MALDI-TOF analysis. However, there is a limitation in detecting a protein of infinitesimal (<100 ng, e.g., <50 ng, <10 ng) quantity in a sample by Coomassie staining.

Meanwhile, although silver staining has been employed in isolating and then detecting a protein of infinitesimal quantity, the method is not compatible with MALDI-TOF analysis. Silver staining is a cumbersome technique to perform. Reproducibility in comparing and analyzing two protein samples analyzed by silver staining is also problematic because the respective samples must be subjected to two-dimensional electrophoresis separately.

The above information disclosed in this Background section is only for enhancement of understanding of the background of the invention and therefore it may contain information that does not form the prior art that is already known in this country to a person of ordinary skill in the art.

The present invention was developed to solve the problems associated with analyzing a protein by previous two-dimensional electrophoresis techniques. The method entitled 2D-DIGE (2D difference gel electrophoresis) was developed that applies a fluorescent material to two-dimensional electrophoresis.

To overcome the limitations of Coomassie and Silver staining methods, 2D-DIGE (2D difference gel electrophoresis) applies a fluorescent material to two-dimensional electrophoresis. The 2D-DIGE method is a technique for performing two-dimensional electrophoresis by employing fluorescent dyes (e.g., Cy3 (green), Cy5 (red), Cy2 (blue) (GE Healthcare Bio-Sciences, Sweden)), which react with a protein on two-dimensional electrophoresis, and qualitatively and quantitatively analyzing the difference in fluorescence, which detects the presence of proteins in an electrophoresed gel, thereby screening two or three samples simultaneously. 2D-DIGE method surpasses the sensitivity of silver staining in analyzing images for a protein of infinitesimal quantity in a sample.

Meanwhile, comparatively more protein (>0.1-10 pmol/μl) is required for identifying a protein by MALDI-TOF. Accordingly, labeling the amount of protein needed for identification by MALDI-TOF with CyDye to analyze by 2D-DIGE results in problems of economy (CyDye is expensive to the degree of 6 million Korean won per 1 mg) and measurement sensitivity (if much CyDye is used, the sensitivity is dropped due to its too strong fluorescence) occur.

Nevertheless, for identification by MALDI-TOF of a protein representing a specific spot on a CyDye-labelled 2D-DIGE technique, a prep gel is prepared separately, to obtain the amount of protein by 2-dimensional electrophoresis that is needed for MALDI-TOF analysis. However, preparation of an additional prep gel is inconvenient and can introduce experimental error and/or lack of reproducibility when compared to a CyDye-labelled 2D-gel .

### SUMMARY OF DISCLOSURE

It is an object of the present invention to provide a method for analyzing a protein, which is highly reproducible and identifies a protein more easily than previous methods. By employing CyDye, multiple (e.g., two, three, four, or more) protein samples can be simultaneously compared and analyzed, and an a protein of infinitesimal quantity can be analyzed. Additionally, it is not required to prepare a prep gel separately to obtain enough protein for identification by MALDI-TOF.

Another object of the present invention is to provide a method for analyzing multiple protein samples in an electrophoresed gel that can detect a difference between a test protein sample and a control protein sample, and simultaneously isolate a spot in the same gel for MALDI-TOF analysis.

In order to achieve the above objects, one aspect of the invention provides a method of detecting a difference in protein distribution between a plurality of protein samples and isolating and identifying a protein different in protein distribution between the protein samples, the method including the steps of: (A) labeling small amounts of each of the protein samples with CyDyes having different fluorescence properties, respectively; (B) mixing 50-100 μg of each of the protein samples, labeled in step (A), with 1-5 mg of each of one or more of the unlabeled protein samples, and subjecting the mixture to electrophoresis; (C) subjecting the electrophoresed gel to fluorescence analysis to detect a difference in protein distribution between the protein samples; and (D) excising spot(s), showing the difference in protein distribution, from the gel, and isolating and identifying a protein, which is different between the protein samples, from the spots.

Labeling using the fluorescent dye in step (A) can be achieved by methods known in the art. Selection of a fluorescent dye to label a first protein group and to label a second protein group can be optionally determined. In an embodiment of the present invention, CyDye is employed, but the selection of CyDye does not influence the result of analysis as demonstrated herein.

The specific process of the two-dimensional electrophoresis, such as preparation of samples, loading of samples, and application of voltage, etc. for the two-dimensional electrophoresis in step (B) can be properly selected from any of the processes well known in the art.

Steps (C) and (D) involve performing the fluorescence analysis on the gel, i.e., the product of electrophoresis, and isolating and extracting a protein from the gel to identify the protein, respectively. These processes can be selected from any of the processes well known in the art.

The analysis of two protein samples and the analysis of three or more protein samples are identical to each other in terms of the concept and practice of the invention. Accordingly, hereinafter, for the sake of convenience of understanding, an explanation is described based on the analysis of two protein samples. In the analysis of two protein samples, the protein samples are referred to as a ‘first protein sample’ and a ‘second protein sample’, respectively.

### BRIEF DESCRIPTION OF DRAWINGS

FIG. 1 is a flowchart showing a process for analyzing a protein according to an embodiment of the present invention;

FIG. 2A-2E are photographs showing the result of 2D-DIGE at pH 4.5-5.5 for a pregnant bovine serum protein according to the present invention compared to 2D-DIGE at pH 4.5-5.5 for a non-pregnant bovine serum protein;

FIGS. 3A-3D are photographs showing the result of 2D-DIGE at pH 6-9 for a pregnant bovine serum protein according to the present invention compared to 2D-DIGE at pH 4.5-5.5 for a non-pregnant bovine serum protein;

FIGS. 4A-4D depict MALDI-TOF analysis showing the identification of protein (spots D1 and C1 shown in FIGS. 4A and 4B, respectively) isolated by protein analysis according to an embodiment of the present invention, and the corresponding MALDI-TOF spectra (FIGS. 4C and 4D); and

FIGS. 5A-5D depict the analysis of spot A1 in FIG. 3D and a MALDI-TOF spectrum showing the identification of a protein isolated by protein analysis according to another embodiment of the present invention.

### DETAILED DESCRIPTION

Hereinafter, the present invention is described in detail with reference to the examples describing screening a protein expressed in pregnant bovine serum. The examples are intended for illustrative purpose, and the present invention is not limited to these examples.

A first protein sample (50-100 μg) labeled with a CyDye and a second protein sample (50-100 μg) labeled with another CyDye, are mixed with 1-5 mg of the first protein sample (unlabeled) only or, additionally, 1-5 mg of a second protein sample (unlabeled) (i.e., a mixture comprising the labeled first protein sample+the labeled second protein sample+the unlabeled first protein sample, and in some cases the unlabeled second protein sample). The mixture is subjected to electrophoresis. By subjecting the electrophoresed gel to fluorescence analysis, \textcircled{1} a protein present in both the first protein sample and the second protein sample (detected in spots showing fluorescence properties of both dyes), \textcircled{2} a protein present in only the first protein sample (detected in spots showing only the fluorescence property of the dye used to label the first protein sample only), and \textcircled{3} a protein present in only the second protein sample (detected in spots showing the fluorescence property of the dye used to label the second protein sample only) are analyzed qualitatively and quantitatively. Herein a small number of proteins are present in spots labeled with a single fluorescent dye among many proteins that are present in spots that are not differentially labeled, and identically present in both protein samples. After analysis, the differentially labeled spots are excised from the gel, and a protein that is different in protein distribution between the protein samples is isolated according to a known method, and then the isolated protein is identified by employing a technique known in the art.

As demonstrated in an embodiment of the present invention, two-dimensional electrophoresis on a mixture of a small amount of a protein sample labeled with a fluorescent dye according to the present invention and a large amount of the unlabeled protein sample resulted in the same 2D-DIGE image as two-dimensional electrophoresis on the fluorescent dye labeled sample only. The fluorescently labeled protein spots also matched the spots of the unlabeled protein samples when stained with Coomassie stain.

According to the present invention, the difference in protein distribution (or expression) between the first protein sample and the second protein sample can be ascertained. That is, any spot in 2D-DIGE analysis showing only the CyDye label of the first protein sample or only the CyDye label of the second protein sample respectively indicates that there is a protein present in the first protein sample only or the second protein sample only.

Further, in the present invention, an internal standard, which is a mixture of the first protein sample and the second protein sample, is labeled with a third CyDye in step (A) and added in step B. The internal standard is subjected to electrophoresis along with the labeled and unlabeled protein samples. Through this process, an error in which the difference in the first protein sample and the second protein sample appears falsely can be avoided (e.g., due to simple contamination or error). That is, a spot for the internal standard is observed at the same location as a spot having a difference in the protein distribution between the protein samples,. However, a spot present in either protein sample because of simple contamination does not display a spot for the internal standard. Thus it is possible to exclude a spot resulting from simple contamination.

According to the present invention, a difference in protein distribution between the first protein sample and the second protein sample can be analyzed by using an automatic picker in step (C), and simultaneously a protein corresponding to a spot showing a difference in protein distribution can be isolated. Further, for the purpose of facilitating isolation, the electrophoresed gel can also be subjected to Coomassie staining between the steps (C) and (D). Coomassie staining facilitates the excision of a corresponding spot region from the electrophoresed gel. In order to isolate a protein of a corresponding spot from the stained electrophoresed gel and to identify a protein, staining is removed according to a known method, and then identification is performed.

The protein isolated by the above step can be used as a sample for identifying a protein. Any method known in the art, for example, MALDI-TOF analysis can be used as a method to identify a protein.

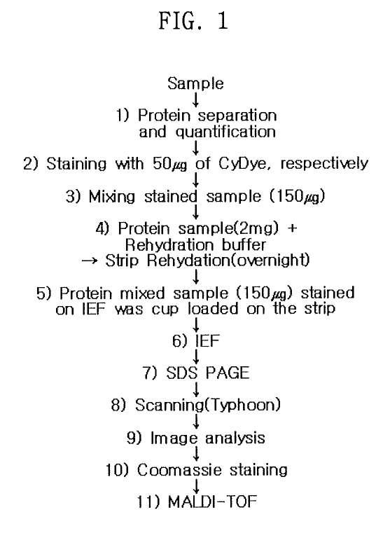

FIG. 1 is a flowchart showing a process for analyzing a protein according to an embodiment of the present invention. However, the detailed order shown in FIG. 1 is only an example, and the present invention is not limited thereto. For example, a protein is easily denatured under a basic condition and is not easily isolated in the first electrophoresis for analyzing a protein. To avoid such problems, a strip is rehydrated and a fluorescent dye labeled protein sample and an additional protein sample are cup-loaded together and subjected to two-dimensional electrophoresis as described in the embodiments of the invention.

As described above, according to the present invention, by performing electrophoresis on a sample labeled with CyDye and an unlabeled sample for MALDI-TOF simultaneously on a 2-D gel, a qualitative and quantitative analysis can be performed with a difference in the fluorescence of CyDye having very high sensitivity of detection, and from the same gel a protein having a different protein distribution between the two samples can be identified using MALDI-TOF. Thus, the limitation of reproducibility can be overcome, and the time and cost for analysis can be remarkably reduced.

Further, because multiple (e.g., two, three, or more) protein samples can be simultaneously analyzed in a gel, the method according to the present invention can be effectively employed when protein samples are compared and analyzed with each other. A protein that has a different distribution between the protein samples is isolated and identified.

The following examples illustrate the invention and are not intended to limit the same.

### Examples

[1] Preparation of a Protein Sample

(1) Protein Isolation from a Serum

In order to isolate a serum protein from a serum, a pregnant serum (200 μl) (21-days after artificial insemination) sample and a non-pregnant serum sample (200 μl) were each mixed with Lysis buffer (200 μl of 1% SDS, 1 mM PMSF, Protease inhibitor cocktail (complete; Roche), 100 mM Tris-HCl pH 7.0). Each mixture was pulverized with a sonicator, and then cooled in an ice bath. The cooled sample was incubated at ambient temperature for more than 30 minutes with shaking, and centrifuged at 15000 rpm, 4° C., and 20 minutes. The resulting supernatant was collected, the amount of protein was quantitated using a commercially available protein quantiation kit (2-D Quant Kit(GE Healthcare Bio-Science)). The supermatamts were divided into aliquots of 2 mg and 50 μg of protein and stored at −70° C. Equal amounts of pregnant serum protein (25 μg) and non-pregnant serum protein (25 μg) prepared in the above process, were mixed to prepare a mixture (50 μg) for use as an internal standard.

(2) Fluorescent Labeling for a Protein Sample

\textcircled{2} For analysis at pH 4.5-5.5, a non-pregnant serum protein sample was labeled with Cy3, a pregnant serum protein sample was labeled with Cy5, and an internal standard was labeled with Cy2 using CyDye DIGE Flors (minimal dyes) kit (GE Healthcare Bio-Science).

\textcircled{2} For analysis at pH 6-9, a non-pregnant serum protein sample was labeled with Cy5, a pregnant serum protein sample was labeled with Cy3, and an internal standard was labeled with Cy2.

[2] Two-Dimensional Electrophoresis

(1) First Electrophoresis (Isoelectricfocusing, IEF)

\textcircled{1} IEF for pH 4-7 and an Acidic Range (pH 3.5-4.5, pH 4.5-5.5, pH 5.5-6.7)

Pregnant bovine serum protein sample (2 mg), not labeled with CyDye, was mixed with rehydration buffer (6M Urea, 2M Thiourea, 4% CHAPS, 0.4% DTT, 2% v/v IPG buffer pH 4-7) to prepare a sample 400 μl in total volume. The mixture was put into an Immobiline pH gradient dry strip reswelling tray to cover 18cm of a pH 4-7 gradient dry strip. Cover oil was placed over the dry strip, and the dry strip was rehydrated for 16 hours.

This strip was set on a multiphor II (Amersham Biosciences), and then a sample-loading cup was set on the acidic side of the strip. Rehydration buffer was mixed with 150 μg of a labeled protein sample comprising 50 μg of a pregnant serum protein sample labeled with CyDye in the above process [1], 50 μg of a non-pregnant serum protein sample, and 50 μg of an internal standard to prepare a sample 110 μl in total volume, The sample was put in a set cup and covered with cover oil. IEF was performed by applying 100,000 Vhr of electricity.

\textcircled{2} IEF for pH 6-9 and pH 7-11

Pregnant bovine serum protein sample (2 mg), not labeled with CyDye, was mixed with rehydration buffer (7M Urea, 2M Thiourea, 4% CHAPS, 2.5% DTT, 10% v/v isopropanol, 5% v/v glycerol, 2% v/v IPG buffer pH 6-9) was mixed to prepare a sample 350 μl in total volume. The mixture was put into an Immobiline pH gradient dry strip reswelling tray to cover 18cm of either a pH 6-9 or pH 7-11. Cover oil was placed over the strip, and the dry strip rehydrated for 16 hours.

This strip was set on a multiphor II (Amersham Biosciences), and then a sample-loading cup was set on the basic side of the strip. Rehydration buffer was mixed with 150 μg of a labeled protein sample comprising 50 μg of a pregnant serum protein sample labeled with CyDye in the above process [1], 50 μg of a non-pregnant serum protein sample, 50 μg of an internal standard, and 2 mg of a unlabeled pregnant bovine serum and a rehydration buffer (7M Urea, 2M Thiourea, 4% CHAPS, 2.5% DTT, 10% v/v isopropanol, 5% v/v glycerol, 2% v/v IPG buffer pH6-9, pH7-11) to prepare a sample 120 μl in total volume. The mixed sample was put into the loading cup, and IEF was performed by applying 100,000 Vhr of electricity.

(2) Second Electrophoresis (SDS-PAGE Electrophoresis)

\textcircled{2} SDS-PAGE for pH 4-7 and an Acidic Range (pH3.5-4.5, pH4.5-5.5, pH5.5-6.7)

After IEF, the strip in process (1) \textcircled{1} was put into 10 ml of TBP equilibration buffer (0.2 mM Tributyl phosphine, 6M Urea, 2% SDS, 375 mM Tris pH8.8, 20% Glycerol, 2.5% Acrylamide), and then equilibrated with soft shaking for 15 minutes. After equilibration, the strip was put into an upper layer of an immobilized 8%-16% gradient gel, immobilized on Ettan DALT twelve Large vertical system (Amersham Bioscience) containing SDS-PAGE running buffer (1.44% Glycine, 0.1% SDS, 0.3% Tris base), and subjected to electrophoresis with 150 mA of electric current for 16 hours.

\textcircled{2} SDS-PAGE for pH 6-9 and pH 7-11

After IEF, the strip in process (1) \textcircled{2} was put into 10 ml of DTT equilibration buffer (1% DTT, 6M Urea, 2% SDS, 375 mM Tris pH8.8, 20% Glycerol, 2.5% Acrylamide), and then equilibrated with soft shaking for 15 minutes. After discarding the DTT equilibration buffer, the strip was equilibrated in 10 ml of Iodoacetamide equilibration buffer (4% Iodoacetamide, 6M Urea, 2% SDS, 375 mM Tris pH8.8, 20% Glycerol, 2.5% Acrylamide), with soft shaking for 15 minutes. After equilibration, the strip was put into an upper layer of an immobilized 8%-16% gradient gel, immobilized on Ettan DALT twelve Large vertical system (Amersham Bioscience) containing SDS-PAGE running buffer (1.44% Glycine, 0.1% SDS, 0.3% Tris base), and subjected to electrophoresis with 150 mA of electric current for 16 hours.

[3] Scanning and Image Analysis

The gel plates after two-dimensional electrophoresis were rinsed with distilled water, and scanned using Typhoon variable mode imager. By performing analysis employing DeCyder software and obtaining statistical data, the protein spots showing a difference in protein distribution or expression were analyzed.

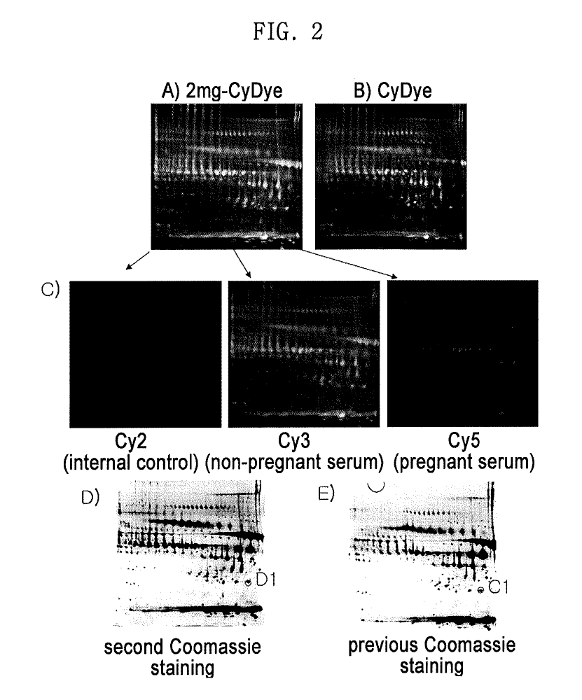

FIG. 2A is a 2D-DIGE image after two-dimensional electrophoresis at an acidic condition of pH 4.5-5.5 in the above process [2] \textcircled{1}, and FIG. 2B is a 2D-DIGE image for a CyDye-labelled sample alone (150 μg) run under the same two-dimensional electrophoresis conditions. The two images above demonstrate that there is no large difference in the isolation profile of the protein spots shown. Further, FIGS. 2C-2E show that protein spots were isolated in the same profile in a non-pregnant serum protein labeled with Cy3 (green) and a pregnant serum protein labeled with Cy5 (red) (FIG. 2C) and an identical gel stained secondly with Coomassie (FIG. 2D).

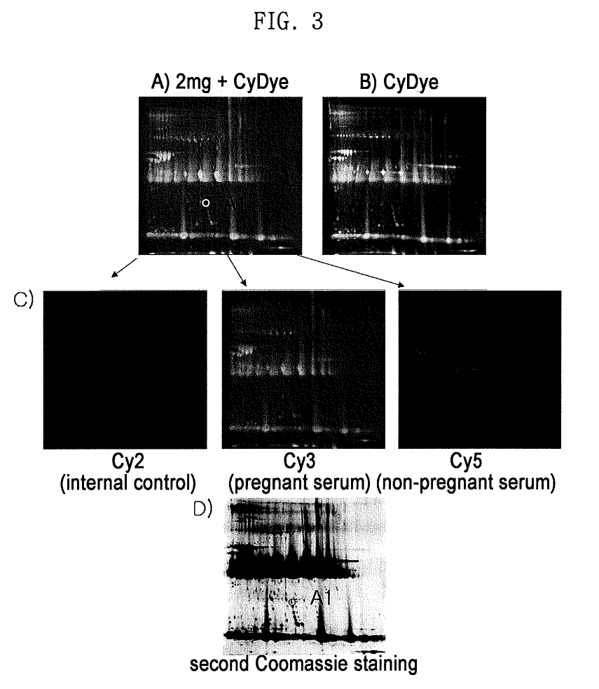

FIG. 3A is an image of 2D-DIGE performed simultaneously for 2 mg of a pregnant bovine serum protein sample and a sample (150 μg) labeled with CyDye by employing pH 6-9 strip in the above process [2] \textcircled{2}. FIG. 3 shows that protein spots were isolated in the same profile in a 2D-DIGE image for a CyDye-labeled sample alone (150 μg) (FIG. 3B), a pregnant serum protein labeled with Cy3 (green) and a non-pregnant serum protein labeled with Cy5 (red) (FIG. 3C), and an identical gel stained secondly with Coomassie (FIG. 3D).

[3′] Staining with Coomassie Brilliant Blue (CBB) G-250

For the case of additionally selecting the step of performing staining with Coomassie blue, the procedure for staining and destaining is as follows.

The gel after scanning was washed with distilled water, placed in 1 l of an immobilizing solution (40% v/v methanol, 5% v/v phosphoric acid), and immobilized for 1 hour with soft shaking.

After immobilization, the immobilizing solution was removed, and 1 l of CBB staining solution (17% g/v Ammonium sulfate, 3% v/v phosphoric acid, 0.1% g/v Coomassie G-250, 34% v/v methanol) was added, and the immobilized gel stained for at least 12 hours with soft shaking.

To destain the stained gel, the stained gel was incubated in destaining solution (1% acetic acid, 0.02% sodium azide) for at least 12 hours.

[4] Protein Identification Employing MALDI-TOF and ESI-MS/MS

The protein spots of interest (spots C1 and D1 in FIGS. 2D and 2E, respectively; and spot A1 in FIG. 3D) were excised (1 mm×1 mm) from the gel after electrophoresis, put into a 1.5 ml microtube. Wash buffer (120 μl; 50% v/v acetonitrile, 25 mM ammonium carbonate, pH7.8) was added. If staining was previously performed, destaining with wash buffer was repeated until the blue color of CBB disappeared. Lyophilization was performed on the excised gel piece by employing a Vacuum centrifuge. The dried gel piece was rehydrated in trypsin buffer (5 μl; 0.02 μg trypsin/ml, 25 mM ammonium carbonate) for 1 hour, 25 mM ammonium bicarbonate buffer was added, and the reaction was performed for at least 12 hours at 37° C. Extraction buffer (5 μl; 50% Acetonitrile, 0.1% Trifluoro acetic acid (TFA), third distilled water) was added, and the sample sonicated for 30 minutes. 1 μl of sample was taken, to which 1 μl of a matrix solution (α-cyano-4-hydroxycinnamic acid 10 mg/g in 50% v/v acetonitrile, 0.1% TFA) was added and mixed, plated on 96-well plate (Perseptive Biosystems, Framingham, Mass., USA), and dried for 30 minutes to prepare a sample. The sample in the 96-well plate was analyzed with MALDI-TOF (Perseptive Biosystems, Framingham, Mass., USA) to measure the molecular weight of a peptide in the sample. The molecular weight data for the measured peptide was searched in a publically available website database (PROFOUND, prowl.rockefeller.edu/prowl-cgi/profound.exe; e.g., as described in Kim et al., Proteomics, 6, 4262-4273, 2006) to identify a protein.

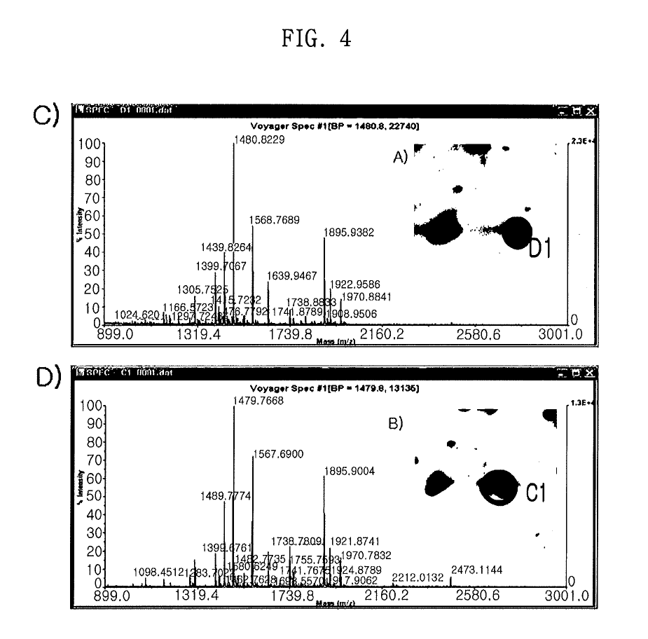

FIG. 4A is a photograph enlarging spot D1 shown in FIG. 2C, and FIG. 4C is a MALDI-TOF spectrum of the protein isolated from corresponding spot by the method of the present example. FIG. 4B is a photograph enlarging spot C1 shown in FIG. 2D, and FIG. 4D. is a MALDI-TOF spectrum of the protein isolated from the spot. From identification results depicted in FIGS. 4C and 4D, it was shown that the peak values in two spectrums are all matched and identified as a serum albumin precursor which is an identical protein.

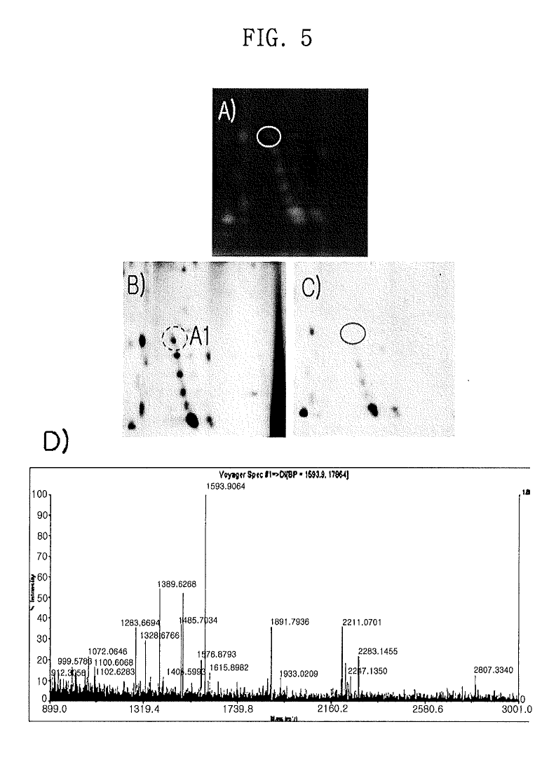

FIGS. 5A-5D show the results of analysis of spot A1 marked in FIG. 3D, FIG. 5A shows a fluorescence analysis, and FIG. 5B shows the result of analysis by Coomassie staining. In FIG. 5A, it can be seen that spot A1 is a protein present in non-pregnant bovine serum only, and not present in a pregnant bovine serum because the spot showed red fluorescence. FIG. 5C is a photograph of the gel stained with Coomassie dye after two-dimensional electrophoresis according to methods known in the art. The protein corresponding to spot Al was isolated by a method of the present example, and analyzed with MALDI-TOF. From this analysis (FIG. 5D), the protein was identified as a Modified Bovine Fibrinogen.

According to the present invention, for example, an protein specifically of infinitesimal quantity expressed in a specific cancer can be identified by comparing and analyzing a serum protein of a healthy person with a serum protein of a specific cancer patient. A kit for detecting the specific cancer, etc. can be manufactured that employs this method of detection, and research on the function of the specifically expressed protein can be applied to the development of an anti-cancer agent.

Although the present invention has been described with reference to several embodiments of the invention, the description is illustrative of the invention and is not to be construed as limiting the invention. Various modifications and variations may occur to those skilled in the art, without departing from the spirit and scope of the invention as defined by the appended claims.

1. A method of detecting a difference in protein distribution between a plurality of protein samples and isolating and identifying a protein different in protein distribution between the protein samples, the method comprising the steps of:
(A) labeling small amounts of each of the protein samples with CyDyes having different fluorescence properties, respectively;
(B) mixing 50-100 μg of each of the protein samples, labeled in step (A), with 1-5 mg of each of one or more of the unlabeled protein samples, and subjecting the mixture to electrophoresis;
(C) subjecting the electrophoresed gel to fluorescence analysis to detect a difference in protein distribution between the protein samples; and
(D) excising spot(s), showing the difference in protein distribution, from the gel, and isolating and identifying a
protein, which is different between the protein samples, from the spots.

2. The method according to claim 1, wherein an internal standard, labeled with CyDye having fluorescence properties different from those of the CyDyes of claim 1, is also added in step (B).

3. The method according to claim 1, further comprising, between step (C) and step (D), a step of analyzing an image by Coomassie staining in order to confirm the spot(s) showing the difference in protein distribution in the gel and to facilitate the excision of the spot(s).

4. The method according to claim 1, wherein the protein identification in step (D) is performed by a MALDI-TOF method.

5. The method according to claim 1, wherein the fluorescence analysis in step (C) is performed using an automatic picker.

6. The method according to claim 2, further comprising, between step (C) and step (D), a step of analyzing an image by Coomassie staining in order to confirm the spot(s) showing the difference in protein distribution in the gel and to facilitate the excision of the spot(s).

7. The method according to claim 2, wherein the protein identification in step (D) is performed by a MALDI-TOF method.

8. The method according to claim 2, wherein the fluorescence analysis in step (C) is performed using an automatic picker.
